(19) United States
(12) Patent Application Publication
(10) Pub. No.: US 2010/0129895 A1
(43) Pub. Date: May 27, 2010

(54) Biofiltration system for odor control

(76) Inventor: Martin Crawford, Stateline, NV (US)

Correspondence Address: William S. Bernheim, 255 N. Lincoln St., Dixon, CA, 95620, US

(21) Appl. No.: 12/313,496

(22) Filed: Nov. 21, 2008

(51) Int. Cl. A62D 3/02; C12M 1/00

(52) U.S. Cl. 435/262.5; 435/283.1

(57) ABSTRACT

The invention involves the creation of a corrosion resistant gas treatment system comprising a unitary housing subdivided into at least two internal sections, the only connection between the first and the following section(s) is a gas only passage channel; the design allows the simultaneous remediation of contaminants from multiply contaminated gas streams in which some of the contaminants require the presence of microorganisms tolerant of widely differing pH and moisture needs for their reduction. The operation is simple and very efficient because the absence of fluid communication between the two sections allows keeping the moisture levels, pHs and microorganism profiles of the two sections specifically optimized for the target pollutants in the gas stream.

### BACKGROUND OF THE INVENTION

1. Field of Invention

The invention relates to an apparatus and process for treating air streams to remove pollutants. More particularly it relates to a system that allows for remediation of multiple contaminants by contaminant-specific remediation organisms having differing pH and moisture needs, the continuous flow process being performed within an internally segmented unitary housing in which there is no fluid connection between the sectional treatment chambers.

2. Description of the Relevant Prior Art

Vaporous pollutants, which are frequently toxic or corrosive or both, are created in a multiplicity of municipal, commercial and agricultural processes and become part of output airstreams. Treatment of these output airstreams to strip out the pollutants is important to human health, to prevent damage to equipment, to protect the environment and to provide odor control.

The earliest methods used to deal with these pollutants were by physical and chemical processes. The physical processes unfortunately created large amounts of contaminated waste materials that then had to be dealt with. The chemical treatment methods that replaced physical decontamination are well established and reliable, however, they involve the use of hazardous chemicals and are associated with the need for increased safety features that increase the footprint and the operating costs of the units.

These drawbacks led to the development of biological treatment of air streams that has proven itself to be effective, safe and cost effective. Such biological treatment systems are capable of treating high flows of contaminated gas having high inlet concentrations of pollutants.

A typical biological treatment system involves passing the contaminated air stream through an inert, porous media base that has been inoculated with and supports the growth of specific microorganisms. The contaminated air passes over the organisms which feed on and convert the pollutants into innocuous compounds, thus removing the odor and other undesirable components and allowing the release of remediated air.

The whole process taking place within a containment structure which serves as a unit. In instances where the air stream contains corrosive gases, the materials used to form the treatment unit are chosen to be as non-reactive as practical. Issues include treatment unit size, the need to deal with multiple contaminants that require different pH and moisture conditions for the support of the bio-organisms that remediate each of the contaminants and providing a control system that provides maximal remediation with minimal complexity.

Some of the most common air stream contaminants include hydrogen sulfide, mercaptans, amines and various organic acids. Hydrogen sulfide gas is toxic and very corrosive and is found in places as diverse as municipal sewage and sewer lines, oil well drilling locations, wood processing plants, and various other municipal and industrial processes in which elemental sulfur comes into contact with organic materials. This is the gas associated with the smell of ‘rotten eggs’. It is very toxic and can kill by asphyxiation, or by explosion.

A very effective method for treating hydrogen sulfide is to pass the air containing hydrogen sulfide through a highly porous, chemically inert media that is being bathed in water at a pH in the range of 1.8-2.2. Under these conditions a biological culture can be made to grow on the media and the cultured organisms will use hydrogen sulfide as a food source, converting the hydrogen sulfide to sulfuric acid using oxygen present in the air.

Organic Compounds other than hydrogen sulfide can be treated by moving the air containing these organic compounds through a highly porous, chemically inert media at a neutral or mildly alkaline pH while ensuring that the air is humidified and the media is kept moist through supplemental irrigation. Under these conditions a biological culture that will use these organic compounds as a food source can be made to grow on the media and convert those compounds to carbon dioxide and other by-products using oxygen present in the air.

Systems that try to simultaneously treat gas streams containing hydrogen sulfide as well as other organic compounds run into the following problem: oxidation of the hydrogen sulfide component of these complex air streams yields a by-product of sulfuric acid that interferes with the development of the biological substrate necessary for the treatment of the non hydrogen sulfide components of the air stream.

Oxidation of hydrogen sulfide takes place primarily at low pH conditions, and requires the use of autotrophic Thiobacillus bacteria. The bulk of the other contaminants commonly encountered in mixed contaminant airstreams require the use of heterotrophic bacteria at close to neutral pH conditions. The presence of both autotrophic and heterotrophic bacteria within a single treatment chamber causes a competition between the various bacteria at the required operating conditions. This in turn leads to reduced efficiency in the system because the non-separated fluid sections do not lend themselves to optimizing the pH in the sections of the treatment unit that are dealing with compounds requiring acid vs. neutral or base tolerant strains of microbial flora. It also leads to the need for using an increasingly complex system of trying to balance the pH of the water to the needs of the differing bacterial colonies within the treatment unit.

Bonnin et al., in U.S. Pat. No. 5,858,768, describe a system for the biodegradation of sulfurous compounds in combination with the physical/chemical elimination of organic nitrogenous compounds. The system is not continuous for the removal of both sets of pollutants, and though alteration of pH is provided for, the pH parameters described do not provide optimal target pH levels for either the acid or the base environment dependent microorganisms, thus likely leading to less efficiency in clearing Hydrogen sulfide gas. Like Horn, U.S. Pat. No. 5,869,323, Koers in U.S. Pat. No. 5,445,660 describes a system in which the polluted air is passed through at least two or more separate housings for purposes of treating pollutants requiring environments of differing pH and moisture for the biologically active components in the chambers. Needing multiple housings increases the cost and the number of connecting elements, pumps, seals and monitoring devices needed and thus would seem to create a less cost effective approach. Parker, et al in U.S. Pat. No. 7,276,366 describe a vertical treatment unit having two media containment sections within a unitary housing. However, the vertically stacked media sections are separated only by the perforated floor of the upper section. Contaminated air enters through an inlet at the bottom of the unit and passes sequentially upwards through both media bed sections and thence out an exhaust stack. Water for moistening the media bed, carrying in microorganisms or altering the pH can be introduced atop either the top section or the lower section in a reverse flow direction to the movement of air in the unit. However, any fluid entering the top section must percolate into and through the lower bed in order to enter the sump and exit the system, this raises the pH in the lower section. A complex, computer controlled system is required to periodically, and for a predetermined run time, alternate between passing fresh irrigating water, or recycling acidifying water from the sump into one or the other or both bed sections in an attempt to maintain pH in the 1.8-2.2 range for optimal clearance of Hydrogen sulfide in the lower (“Bioscrubber”) section. The upper (“Biofilter”) section pH being controlled in a similar manner such that it suits more alkaline loving microorganisms. Having a fluid connection between the bioscrubber and biofilter sections of the treatment unit leads to increased complexity of the control system and decreased specificity of the pH levels for optimal colonization of the microorganisms in the two sections of the treatment unit. As with any such media bed system, the vertical height is limited by the need to prevent compaction of and channel formation within the media beds. A series of these vertical units would be needed to handle larger volumes of contaminated air, resulting in the need for additional computer control systems which of course leads to a higher cost for the system.

Past designs for systems capable of remediating air streams containing mixed pollutants have suffered from the need to use multiple units for large scale operations and that led to increased installation costs. The fluid connection between the sections of the treatment units created alterations of pH that reduced the units' decontamination cost-efficiency. Some have required complex control systems to try and maintain proper pH, moisture levels and microbial populations tolerant of the pHs of the varying pollutants in the air stream because of the fluid communication between the internal sections of the unit.

### Statement of the Objectives

Accordingly, it is an objective of this invention to provide a unitary housing treatment Unit having no fluid connection between its two or more, separated, fluid containing, internal treatment chambers.

A further objective is to provide a unitary, corrosion resistant, housing that can be created having the structural strength allowing for its use in large commercial and municipal reactors, yet also having a flexibility of design allowing for use in small sized reactors.

Another objective is to provide a treatment unit that allows optimal control of pH, moisture levels and microbial population purity within the separated internal sections of the structure that are intended to separately and sequentially remediate Hydrogen sulfide, which requires microorganisms that are very acid tolerant, and other pollutants such as mercaptans, amines and various organic acids that are dealt with by microorganisms that can only flourish at neutral or basic pH levels.

Another objective is to provide a system that is easily managed and largely self-regulating thus reducing operating costs.

### SUMMARY OF THE INVENTION

The invention involves the creation of a unitarily housed air treatment system for the remediation of mixed air stream pollutants that embodies at least two separate treatment chamber sections, at least one of which requires the presence of microorganisms tolerant of a highly acidic pH while another or other sections require a neutral or basic pH. The design allows complete independent control of media bed pH, moisture levels, and microorganism population types within the two treatment chamber sections. The air pathway is from below upwards with countercurrent moisture application from above down in both treatment chambers. The only connection between the first and the following section(s) is an air-communication only channel which allows air that has passed through the more acidic treatment chamber wherein Hydrogen sulfide and like products are reduced, to move into the Unit's further chamber(s) without altering the moisture and pH thereof. Sensors and regulators are used to keep pH, moisture levels and rate of flow within predetermined parameters in both treatment chambers.

### BRIEF DESCRIPTION OF THE DRAWINGS

FIG. 1. Presents a longitudinal cross sectional view of a treatment unit. The drawing is not to scale, the vertical component having been expanded for clarity.

FIG. 2 Presents a transverse sectional view of the same unit looking down from above, but with the roof section removed. The drawing is not to scale, especially as regards the width which is exaggerated in respect to the length of the unit

FIG. 3. Presents a detail of the air-communication only channel that separates the first treatment-chamber (“bioscrubber”) and second treatment-chamber (“biofilter”) sections of the treatment unit.

FIG. 4 Presents a diagrammatic representation of the control system, electrical and water supplies and the waste water removal system of the treatment unit.

### DETAILED DESCRIPTION OF A PREFERRED EMBODIMENT

Further objectives advantages and novel features of the invention will be apparent to those skilled in the art from the following detailed description when taken in conjunction with the accompanying drawings illustrating a preferred embodiment of the invention.

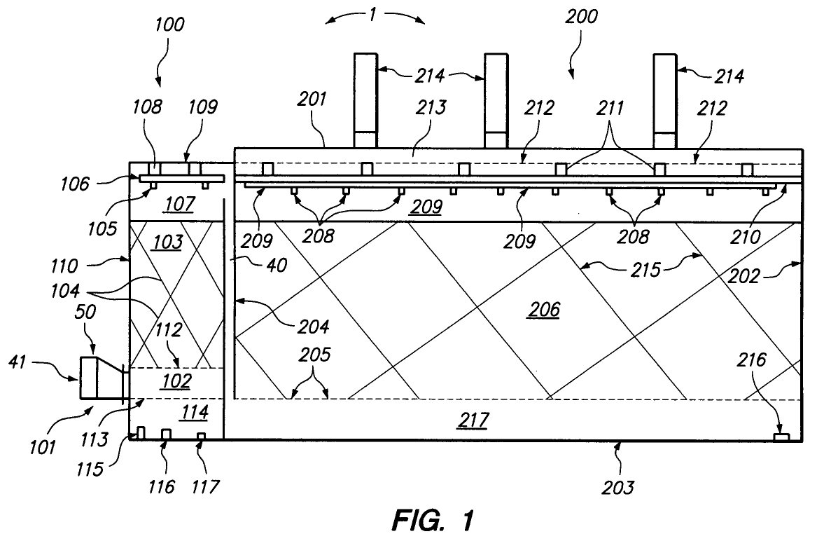

As seen in FIG. 1, the invention involves the creation of a unitarily housed gas stream purification treatment unit (“Unit”) 1 fabricated from fiber glass reinforced plastic (FRP), or other chemical resistant material such as fiberglass, plastic, stainless steel; the Unit 1 being internally divided into at least two treatment chambers having no fluid connection, a bioscrubber (“Scrubber”) section 100 and a biofilter (“Filter”) section 200 that are connected by a gas communication only channel (Gas Channel) 40;

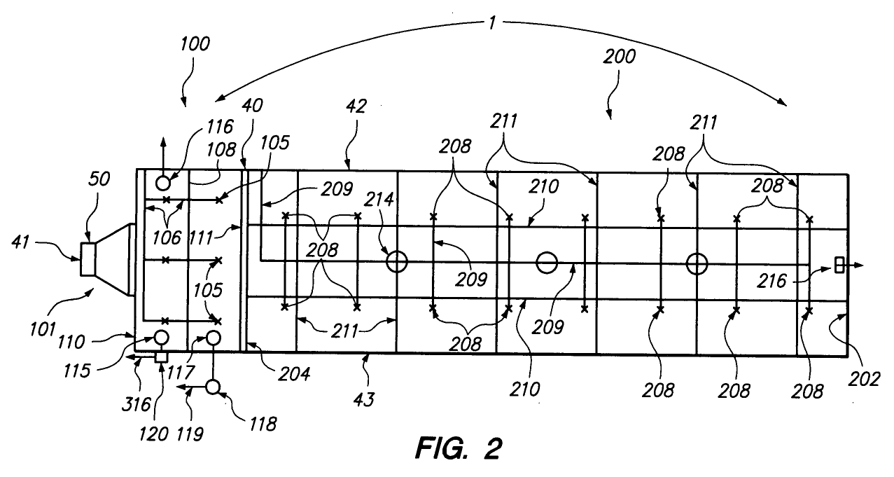

Gas Channel 40 creates a non-fluid connection between Scrubber and Filter sections 100 and 200; the totality of the treatment Unit 1 externally comprising a front wall 110, a Scrubber section 100 roof 109; a dome shaped Filter section 200 roof 201, a rear wall 202 and a floor 203; the whole being fabricated of FRP, or of FRP reinforced by steel support members (none depicted) that are sealed away from the internal chambers of said Unit by being coated with a chemical resistant material when issues of size and weight dictate such reinforcement. Note: a pair of side walls 42 FIG. 2 and 43 FIG. 2 are not indicated as such in this view.

Continuing with the view shown in FIG. 1, a contaminated gas stream enters the Scrubber section 100 via the driving force of a fan (not shown), the gas stream flows in the direction indicated by the arrow 41 and thus into an inlet flange assembly 101 that is affixed to the Scrubber section 100 front wall 110; the gas then moves into a gas stream entry plenum 102; a perforated plate 112 delineates the top boundary of the plenum 102 and also serves as the bottom limit of a media section 103; the lower limit of the gas stream entry plenum 102 is formed by a perforated plate 113 which also serves as the top boundary of a sump 114 of Scrubber section 100; the gas stream next moves up into media bed section 103 after which it passes into a moisturization chamber section 107 where it passes beneath a series of sprinklers 105 located on a set of water lines 106 that are attached to a series of FRP cross braces 108 which in turn are affixed to the undersurface of the roof 109 of Scrubber section 100;

The gas stream, now cleansed of some contaminants moves up and over the Scrubber section 100 rear wall 111, which will be noted extends below to the floor 203 and ends above at a short distance beneath the Scrubber section 100 roof 109, forming the front wall of the Gas Channel 40; a front wall 204 of the Filter section 200 forms the rear wall of Gas Channel 40; the Filter section 200 front wall 204 depends down from the Filter section 200 domed roof 201 and ends below at a perforated floor plate 205.

Perforated floor plate 205 serves as both the base of the Filter section 200 media bed 206 and as the top plate of the Filter section 200 sump section 217; the Filter section 200 sump 217 is drained by overflow drain 216 that allows water collected therein to be evacuated such that the sump 217 contains a headspace which serves as an entry plenum into which the gas stream being treated passes from Gas Passage 40.

Following which the gas stream moves up through perforated floor plate 205 into the Filter section 200 media bed section 206, then up into a moisturization chamber section 207 where supply water is added by a series of sprinklers 208 attached to a water inlet line array 209 FIG. 1 (best seen in FIG. 2) that is supported along a pair of longitudinal FRP supports 210 which in turn are affixed to a series of FRP cross supports 211 that are attached to the Filter section 200 perforated, internal top plate 212.

The Filter section 200 perforated internal top plate 212 also serves as the floor of a gas stream exit plenum 213 where the purified gas collects and then moves up into a set of exhaust stacks 214 and finally into the ambient air mass.

Scrubber section 100 media bed section 103 contains an inert media 104 (cross hatching), such as conventional foam, reticulated foam, plastic or other such acid resistant synthetic materials; whichever media material is selected for use, that material is inoculated with and serves as the support substrate for colonies of autotrophic micro-organisms that feed on Hydrogen sulfide gas, the primary component of the gas stream removed in Scrubber section 100; less quantitavely prominent organic sulfides, ammonia, amines and such compounds are also removed in the scrubber 100 media bed 103.

Filter section 200 media bed section 206 contains an inert medium 215 (cross hatched area) such as granulated carbon, other carbon based media, wood chips, engineered media, lava rock or other such media that are inert to mildly alkaline solutions; whatever the media type selected for use, the media material is inoculated with and serves as the support for heterotrophic microorganisms that thrive in a neutral to mildly alkaline environment. These heterotrophic organisms digest organic nitrogenous compounds and other residual contaminants, thus removing them from the gas stream.

Note, the use of the terms “Scrubber” and “Filter” in the preceding and following text refers to two sections of the treatment Unit 1 that are designed to operate on differing component compounds of a multiply contaminated gas stream. Both use microorganisms colonized on base media for purposes of gas stream remediation. Neither section relies on a physical “filtration” system of purification. In all instances, the term “Scrubber” refers to the first air treatment chamber, which is kept at a low pH range, optimally pH 1.8 to 2.2, whereas the term “Filter” refers to the second air treatment chamber which is kept at a neutral to mildly alkaline pH range.

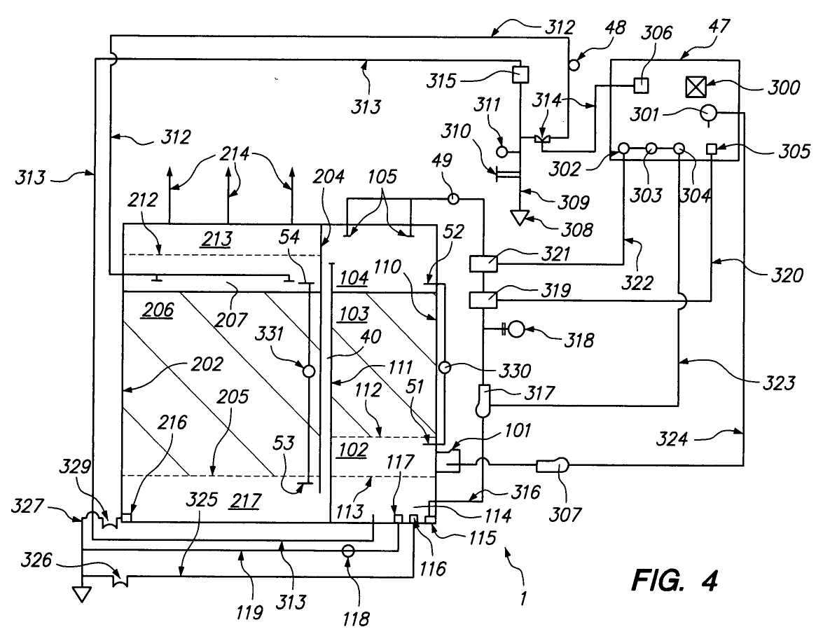

The microorganisms 104 and 215 respectively in the Scrubber and Filter 100 and 200 media bed sections 103 and 206 require a moist environment; moisturization is provided by sprinkler sets 105 and 208, the flow of water from which creates a counterflow movement of water down though the media beds 103 and 206; initially, the water entering both the moisturization chambers 107 and 207 is fresh inlet water from an outside source (not shown); that water having first been conformed to a specific pH range by a control system (not shown) that will be described later and presented diagrammatically in FIG. 4.

pH regulated water from an external source continues to be the only moisturizing water used in Filter section 200 as long as the treatment Unit 1 is in operation. However, the digestion of hydrogen sulfide gas in the Scrubber section 100 leads to the formation of Sulfuric acid that mixes with and increases the acidity of the water to an undesirable pH as it passes down though the Scrubber section 100 media bed 103; this problem is corrected as follows: the hyper acidulated water passes down through air plenum 102 and then through perforated plate 113 into sump 114; some of the hyper acidulated water passes out of the system through a scrubber overflow drain 116; fresh water from an external source (not shown) is mixed in with the remaining hyper acidulated water in Scrubber section 100 sump 114 in order to bring the water into the proper pH range of 1.8 to 2.2, following which pH modification, the water is re-used in the Scrubber section 100 moisturization chamber 107.

No water is recirculated through the Filter section 200 media bed 206 which requires a neutral to alkaline pH and the water flowing into sump 217 passes through overflow drain 216 and is disposed of via the external drain system (not shown).

A Scrubber section 100 drain 117 (best seen in FIG. 2) is controlled by a valve 118 FIG. 2; when fully emptying Scrubber 100 is indicated, water exiting this drain passes into a waste water line 119 FIG. 2 and thus out of the system.

When viewed from above as in FIG. 2, some further aspects of the invention, and other spatial aspects of features seen in FIG. 1 can be viewed. A brief review of the dynamics of the working of the system follows for the purpose of orienting the process within this view looking down into the Unit 1 with the roof sections 109 FIG. 1 and 201 FIG. 1 removed.

Thus, in FIG. 2 side walls 42 and 43 of the treatment Unit 1 are now seen completing the perimeter shell along with front wall 110 and rear wall 202; a recirculation pump 120 is affixed to the Scrubber section 100 recirculation drain 115; a recirculation system water line 316 (best seen in FIG. 4) passes out from recirculation pump 120 and forms part of a recirculation and control system that will be described later.

A contaminated gas stream enters from a source 41 and after passing from a contaminated gas stream inlet duct (not shown) that is attached to a flange 50, the gas passes through air inlet 101 and thus through the Scrubber section 100 as described prior. Gas Passage 40 is visible between the Scrubber section 100 rear wall 111 and the Filter section 200 front wall 204.

The topmost layer of the moisturizing support and delivery arrangement comprises: two cross braces 108 in the Scrubber section 100, and six cross braces 211 in the Filter section 200. In Filter section 200, longitudinal support beams 210 are affixed beneath the six cross braces 211 and the water line 209, comprising a central pipe with eight laterals, each of which terminates in a sprinkler 208 at both ends, is suspended beneath the longitudinal support beams 210 at each lateral offshoot of the sprinkler line 209.

The moisturizing support and delivery arrangement in the scrubber section 100 differs in that no longitudinal support beams are needed because of its short depth. The Scrubber section 100 water pipe 106 with its sprinklers 105 is suspended solely from the paired cross braces 108 to which it is attached.

For purposes of further orientation in FIG. 2, three circles representing the tops of exhaust stacks 214 that are spaced above the roof (not shown) are seen spaced along the longitudinal center of the Filter section 200; the Filter section 200 overflow drain 216 located in the floor 203 is seen centrally at the rear of the filter section 200.

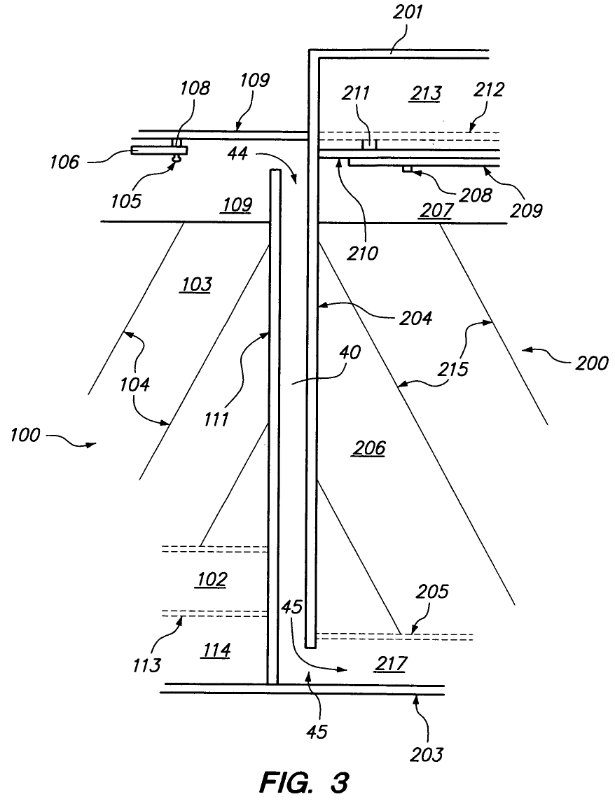

Gas Channel 40, as shown in greater detail in FIG. 3 provides a better understanding of the invention's method of allowing a unitary housing to contain two separate fluid-containing gas stream treatment chambers each of which operates at a separate pH level without fluid connection between the two chambers.

Gas Channel 40 is formed anteriorly by the Scrubber 100 back wall 111 that is integrally attached to the floor 203 and ends above at a distance short of the roof 109; back wall 111 is integrally attached laterally to the right and left side walls 42 and 43 of Unit 1.

Contaminated gas enters the Scrubber section 100, and after passing through media section 103 and into moisturization chamber 107 as partially treated gas, the gas stream then follows the pathway shown by the arrow 44 and passes over the Scrubber back wall 111 and then downwards through Gas Channel 40.

The media bed 103 of Scrubber 100 terminates short of the top of the Scrubber section 100 back wall 107, and in conjunction with the Scrubber 100 overflow drain 116 that prevents excess buildup of exiting water, helps to insure that no water flows from the Scrubber section 100 into the Gas Channel 40 despite the constant counterflow of water entering the Scrubber 100.

Filter section 200 front wall 204 forms the back wall of Gas Channel 40 and is integrally attached above to the Filter section 200 roof 201 and side walls 42 and 43, ending below a short distance from floor 203; thus presenting a space through which the on-moving gas stream, following the direction indicated by arrow 45, turns into the Filter section 200 combination fluid sump/gas stream entry plenum 217; the gas stream then moves upwards through the Filter section 200 media bed 206, etc. as described prior.

As described prior, overflow drain 216 removes excess water from the Filter section 200 sump 217 and sends it into a waste water drain, thus preventing a backup of the treatment water from the Filter section 200 into the Gas Channel 40.

A control panel (“Panel”) 47 FIG. 4 houses the electrical and electronic components that control the electrical power cutoff, gas stream entry fan, moisturizing, recirculation and pH management systems; the Panel presents with a main power switch 300; a fan switch 301; a recirculation pump indicator light 302; a flow relay 303; a power switch 304; a pH meter 305 and a timer 306. With the main power switch 300 turned on, the treatment Unit 1 is ready to operate. Note: because the actual flow patterns of the gas and moisture streams as well as most of the structural components of the treatment unit have already been described, only descriptive text related to pH management, moisture levels and such functional considerations follows.

Continuing with the view presented in FIG. 4, when fan switch 301 is activated electrical current passes on into an electrical power wire 324 to a gas stream inlet fan 307 which then drives the gas stream into the treatment Unit's 1 gas stream inlet 101; note, the fan can alternatively be mounted in the incoming ductwork outside said treatment unit (not shown), or housed within the treatment Unit's 1 gas stream inlet 101.

Water from an external source (not shown) enters via a water inlet valve 308 and thus into a fresh water line 309 where it passes through a pressure regulator 310 then a pressure gauge 311; at this point the water line 309 splits into two separate supplies, one line, a Filter section 200 inlet water line 312, which always delivers only fresh water, passes through a solenoid valve and its associated electrical control wire 314 that is activated by the timer 306 located in control panel 47; timer 306 is set to intermittently spray into the Filter section 200 moisturization chamber 207 using the Filter section 200 sprinkler sets 208 described prior; a port and valve 48 arrangement is located on inlet water line 312 for adding inoculation material, nutrients and other such agents to the Filter section's 200 media bed 206.

The second branch off from fresh water line 309 is a make up water line 313 which first passes through a rotameter 315; the rotameter 315 is adjusted after use and trial to provide a pre-set, stable rate of flow of water to the Scrubber sump 114 from whence the water is then drawn into a recirculation system water line 316 by a recirculation pump 317 which is activated when pump power switch 304 is set in the on position and the signal from pump power switch 304 is carried to the recirculation pump 317 via an electric power line 323; after passing through the recirculation pump 317, the water passes by a pressure gauge 318 then past a pH probe 319 that sends a signal via an electrical control wire 320 to the pH meter 305 in control panel 47; continuing past the pH probe 319, the water passes through a flow transmitter 321 the signal from which passes via an electrical control wire 322 sequentially into indicator light 302, flow relay 303 and thus to pump power switch 304. Flow transmitter 321 serves as a fail safe device and should the water level in the system fall below a critical level, the flow transmitter's 321 altered signal intensity reaches the flow relay 303, which in turn will trip the pump power switch 304, thus turning off the recirculation pump 317 and preventing damage to same; having passed by flow transmitter 321, the water next passes a port and valve 49 located on recirculation line 316; port and valve 49 serve to allow addition of inoculant material, nutrients and other such agents to the scrubber's 100 media bed 103 as needed; finally, the pH corrected water is delivered to the Scrubber section's 100 internal sprinklers 105, the placement of which was described prior.

Because the Scrubber section 100 uses water from the Scrubber sump 114 mixed with some fresh water to maintain an optimal pH in the Scrubber section 100 media bed 103, provision is made for some excess water to escape via an overflow drain system comprising an overflow drain 116 and an overflow drain line 325. Overflow drain line 325 has a trap 326 for the prevention of back flow into the Scrubber section 100 sump 114.

Both Scrubber section 100 and Filter section 200 sumps 114 and 217 have access to a main drain line 327 FIG. 4 that exits into a general drain line (not shown); should need arise to drain the system entirely, opening valve 118 drains the Scrubber sump 114; Filter drain 216 is continuously open and has a trap 329 to prevent backflow into the Unit 1, although an optional valve closure could be used to eliminate the need for provision of a trap on drain line 327.

Note: although they are not part of the control system, exhaust stacks 214 (represented by arrows indicating the final direction of the gas stream flow) are shown for purposes of orientation.

A differential pressure gauge array 330, for the Scrubber section 100, which serves to register the pressure differential at the inlet and exit peripheries of the media bed 103, comprises an externally visible differential pressure gauge attached to a pair of pressure sensitive probes 51 and 52, one of which probes 51 is situated in Scrubber 100 air entry plenum 102 and the other of which probes 52 is situated in the moisturization chamber 107, thus bracketing the media chamber 103 of the Scrubber 100 and allowing determination of and serving warning of bed-compaction or other such problems if the inlet and exit pressure differential becomes too great.

A differential pressure gauge array 331, for the Filter section 200, which serves to register the pressure differential at the inlet and exit peripheries of the media bed 206 comprises an externally visible differential pressure gauge having a pair of pressure sensitive probes 53 and 54, one of which probes 53 is situated in Filter 200 air entry plenum 217 and the other of which probes 54 is situated in moisturization chamber 207, thus bracketing the media chamber 206 of the Filter 200 and allowing determination of and serving warning of bed-compaction or other such problems if the inlet and exit pressure differential becomes too great.

I claim:

1. A device for treating a multiple contaminant polluted gas stream to reduce the concentrations of said multiple pollutants by moving a continuous flow of such a gas stream through a treatment unit comprising a unitary external shell housing at least two sequentially placed fluid containing treatment sections that are separated by an internal pair of intervening partial height walls to form a first treatment section, and a second treatment section; said partial height walls having a space between them, and which said partial height walls being situated with respect to the external shell in a manner intended to form a gas communication only channel situated between said first treatment section and said second treatment section;
said treatment unit's external shell comprising a floor, a roof, a right and a left side wall, a treatment unit front wall, a treatment unit end wall, a gas stream inlet assembly piercing said front wall of said treatment unit through which a multiply polluted gas stream passes into said first treatment section of said treatment unit;
a series of exhaust stacks projecting upward from a series of matching exhaust ports in said roof over a gas exit plenum situated at the top of said second treatment section of said treatment unit through which said treated gas stream passes out into the environment;
said partial height wall at the rear of said fluid filled first treatment section being integrally connected with said floor and said side walls of said treatment unit and extending upwards from said floor to terminate at a location slightly below said roof of said treatment unit; and, said second partial wall, being located a short distance to the rear of said first partial wall and forming an internal front wall of said second treatment section, being integrally attached to said roof and said side walls of said treatment unit, but depending downwards from said roof and terminating at a location slightly above said floor of said second treatment unit; the staggered tops and bottoms of which said partial internal walls forming part of a functional arrangement allowing for passage of said on-flowing gas stream from said first treatment section to said second treatment section without fluid transfer between the two said treatment sections;
means existing within said first treatment section to prevent said first treatment section's partial height rear wall from acting as a weir as a counter flow moisture stream passes downward through a media bed containing an appropriate media, said media in said bed containing microorganisms that remediate some of the pollutants in said air stream;
means existing in said second treatment section for preventing the fluid in said second treatment section from passing backwards and up into said open space between said partial walls as a counter flow moisture stream passes downward through a media bed containing an appropriate media within said second treatment section; said media in said second treatment chamber's treatment bed containing microorganisms that remediate other classes of pollutants than were remediated in said first treatment section;
means existing for controlling an electrical power, water delivery and waste water removal array of operational components of said treatment unit.
means existing for separately monitoring the air pressure differential between the inlet and outlet sides of said media beds in said first treatment section and said second treatment section.

2. The Unit of claim 1 wherein the water stream entering said first treatment section's said treatment bed, has been pre-modified and stabilized within the pH range of 1.8 to 2.2, by the proportional pre-mixing of fresh water from an external source with hyper-acidulated water that has passed through said first treatment section's said treatment bed and into said sump of said first treatment section.

3. The treatment unit of claim 1 wherein, the unitary housing is internally subdivided into more than two treatment sections, a first treatment section being followed by a series of successive secondary treatment sections; said successive secondary treatment sections being separated each from the preceding said successive secondary treatment section by paired, internal, partial height walls; said paired partial height walls having a space between them, and which said partial height walls are situated with respect to the external shell of said treatment unit in a manner intended to form a gas communication only channel situated between a forward situated secondary treatment section and a successor secondary treatment section;
said first wall of any of said pairs of partial height walls that are separating a pair of said fluid containing successive secondary treatment sections, being integrally connected with said floor and said side walls of said treatment unit and extending upwards from said floor to terminate at a location slightly below the roof of said treatment unit; and, said second partial wall of any of said pairs of partial walls being integrally attached to said roof and said side walls of said treatment unit, but depending downwards from said roof and terminating at a location slightly above said floor of said treatment unit; the staggered tops and bottoms of which said pairs of partial internal walls form part of a functional arrangement allowing for passage of said on-flowing gas stream from a forward situated secondary treatment section to a successive secondary treatment section without fluid transfer between the two said sections;
means existing within said first treatment section to prevent said first treatment section's partial height rear wall from acting as a weir as a counter flow moisture stream passes downward through a media bed containing an appropriate media, said media in said bed containing microorganisms that remediate some of the pollutants in said air stream;
means existing in said multiple successive secondary treatment sections for preventing the fluid in any of said successor secondary treatment sections from passing backwards and up into said gas only channel between said partial walls and thence into the preceding secondary treatment section as a counter flow moisture stream passes downward through a media bed containing an appropriate media within said successor secondary treatment section; said media in said successive secondary treatment beds containing microorganisms that remediate other classes of pollutants than were remediated in said first treatment section, and in certain instances being capable of remediating other classes of pollutants than were remediated in the immediately preceding successive secondary treatment section;
means existing for controlling an electrical power, water delivery and waste water. removal array of operational components of said treatment unit.
means existing for separately monitoring the air pressure differential between the inlet and outlet sides of said media beds in said first treatment section and any of the successive secondary treatment sections.

4. A Process for treating a multiple-contaminant polluted gas stream by passing said gas stream through a remediation unit comprising a unitary external shell housing two or more sequentially placed fluid containing treatment sections. The first of which pair of treatment sections are separated by an internal pair of intervening partial height walls to form a first treatment section and a second treatment section; succeeding treatment sections, if any, comprising successive secondary treatment sections, each of which successive secondary treatment sections are separated by a pair of intervening partial height walls, excepting a final chamber which said chamber has a full height rear wall; said process being accomplished by:
a. moving a fan driven gas stream at slightly above ambient air pressure into an inlet manifold of said first treatment section then into a gas stream entry plenum of said first treatment section and vertically upwards through a media bed filled with an appropriately moistened inorganic medium such as foam or reticulated foam that has been inoculated with autotrophic microorganisms tolerant of a pH range of 1.8 to 2.2, which said microorganisms feed on and remove hydrogen sulfide gas, other sulfides, ammonia, amines and such compounds from said airstream, thus partially remediating said gas stream, the while a continuous counterflow moisture stream is passing downwards through said media bed from sprinklers situated above said media bed;
b. means existing in said first treatment section to ensure that fluids do not accumulate in a sump section at the bottom of said first treatment section, thus preventing a weir action of fluid over a partial height rear wall of said first treatment section and facilitating the forward and upward movement of said incoming pollutant laden gas stream; after passing through said media bed in said first treatment section, said partially remediated gas stream continues to pass upwards into a combined gas space/moisturization chamber and thence, driven by the pressure behind it, next moves over said partial rear-wall of said first treatment section; said rear wall being integral with a floor and a pair of side walls, and extending upwards from said floor to a point located a short distance below a roof of said first treatment section;
c. said partially remediated air stream then passes over said first treatment section's partial rear wall moving thus into a gas communication only channel, the rear wall of which said gas communication only channel comprises the external surface of a front wall of said second treatment section, or of any of a series of successive secondary treatment sections, which said front wall extends downward from said roof of said treatment unit and ends a short distance above said floor of said treatment unit, allowing said on-moving gas stream to flow into a gas entry plenum at the bottom of said second treatment section, or of any of said successive secondary treatment sections;
d. where means in said second treatment section, or in any successive secondary treatment section ensures that fluids in said second or said successive secondary treatment sections do not accumulate and pass backwards into said gas communication only channel or move upwards into a media bed of said treatment section and which facilitates the forward movement of said partially remediated gas stream upwards vertically through said media bed; which said media bed contains an organic medium such as granulated carbon, wood chips, other carbon based media, engineered media, lava rock or such other suitable media that has been inoculated with heterotrophic microorganisms tolerant of a neutral or slightly basic pH and which said organisms serve to remove organic nitrogenous compounds and other residual compounds from said contaminated gas stream; said media being continuously fed by a counterflow moisture stream that serves to further remediate said gas stream; and which said moisture stream optionally can carry inoculant or other such emendatory elements into said media bed(s).
e. said gas stream having moved upwards through the properly pH regulated media of said second, or, successive secondary treatment sections' media bed, next passes into a combined gas space/moisturization chamber of the second treatment section and then moves upwards into a gas exit plenum, finally exiting into the ambient environment via a series of exhaust stacks that are situated over said second treatment section; or, in the presence of multiple successive secondary treatment sections, said airstream passes by the same process from each said preceding to each said succeeding sections(s) until reaching the last of said sections and thence moving upwards into said gas exit plenum, and thence into the ambient environment.
